(19) United States
(12) Patent Application Publication
(10) Pub. No.: US 2005/0255180 A1
(43) Pub. Date: Nov. 17, 2005

(54) Prevention of diabetes by medicinal plants & fruits

(76) Inventor: Gaurav Tewari, San Antonio, TX (US)

Correspondence Address: DR. GAURAV TEWARI, 7300 BLANCO ROAD, SUITE 610, SAN ANTONIO, TX, 78216, US

(21) Appl. No.: 11/129,181

(22) Filed: May 13, 2005

Related U.S. Application Data
(60) Provisional application No. 60/571,164, filed on May 14, 2004.

(51) Int. Cl. A61K 35/78

(52) U.S. Cl. 424/725

(57) ABSTRACT

Syzigium cumini, (common name, Jamun) is a preventive herbal medicine, which improves the health of a diabetic patient at initial stage by decreasing the blood sugar level. The invention is related to determine an appropriate dosage to human patients suffering different forms of diabetes. The invention involved efficacy of Jamun extraction on human patients and to show the level of effects in reducing diabetes and to prepare a blend that can be medically administered to diabetic patients with no side-effect. An oral administration of a dose of dried powdered form of Jamun in the range of 5-10 gm atleast twice a day for a minimum of 6 months is required to prevent diabetes.

This application claims the benefits of United States Provisional Application No. 60/571,164.

### TECHNICAL FIELD

This application is related to prevention of diabetes through medicinal fruits.

### BACKGROUND OF INVENTION

A broad search of Ayurvedic medicine/therapies showed that there is sufficient literature to support a systematic review of the use of Ayurvedic therapies for the treatment of diabetes. Diabetes is a common illness, and many traditional medical systems have developed strategies to treat this condition. The Ayurvedic therapy most commonly used to treat diabetes was herbal therapy, which therefore became the primary focus of this review. A small number of studies looking at diet therapy were also found.

Based on the reviewed literature, it was concluded that no concerted effort has been done to fully exploit the potential of tropical fruits and plants to quantify their efficacy in treating diabetes among human beings and to determine a dose required to prevent diabetes.

Empirical evidence of efficacy for the Ayurvedic treatment of diabetes would be helpful to health care providers managing diabetic patients and would be useful in identifying areas for future research. Following information can be readily found in literature:

1. Ayurvedic therapies have been reported in the literature, for different conditions/body systems, and using different research designs.

2. There is evidence to suggest that the single herbs Coccinia indica, holy basil, fenugreek, and Gymnema sylvestre and the herbal formulas Ayush-82 and D-400 have a glucose-lowering effect and deserve further study. Evidence of effectiveness of several other herbs is less extensive (C. tamala, Eugenia jambolana, and Momordica charantia).

3. Different other tropical fruits and plants have the potential for several health benefits, however, no concerted effort is done to examine their efficacy in treating diabetes.

This invention relates to a determination of a dose required of Syzigium cumini, commonly known as ‘jamun’, in treatment of diabetes among human beings.

### SUMMARY OF THE INVENTION

Treatment of Diabetes by Syzigium Cumini (Jamun)

Syzigium cumini, (common name, Jamun) is generally grown in India and it requires international acquaintance due to its high medicinal value. It may be grown at temperatures ranging from 12 to 47 degree Celsius, i.e., under sub-tropical climate successfully. In India, Jamun is available from June to July in abundance. During the short span the fruits are spoiled. The fresh fruits as well as its stone have high medicinal value. It is highly anti-diabetic. The fruit is highly perishable and losses its freshness within 24 hours. The capsules for human consumption can be prepared from the stones of the fruits by blending with the plant product having high vitality properties.

JAMUN, an indigenous fruit of India, belongs to family myrtaceae. It is also known as Indian black cherry.

JAMUN is a rich source of minerals constituents particularly iron; calcium and Phosphorus. Vitamin A and C content of JAMUN fruit are higher than apple. The ripe fruit is astringent, stomachic, carminative, antiscorbutic of the spleen, chronic diarrhoea, heart and lever trouble and scanty urine.

Jamun fruits is highly perishable and seasonal in nature. Considerable losses occur in the fruit during harvesting. There is no standard technique for shelf life and packing of fruits.

The therapeutic value of this fruit lies in an untapped potentiality for medical and food-processing industry. Jamun seeds contain combolian glucocide. This elements creates obstacle in the conversion of starch in to sugar. Beside the combolian glucocide, it also contains chlorophyl, zerbirgin, albumin, gallic acid and other coloured materials.

Although, JAMUN has been used traditionally for a lot of medical and health benefits. Nevertheless, a concerted effort to provide medical and scientific evidence re anti-diabetic properties of Jamun is not reported.

The present invention thoroughly tested different doses of Jamun for treatment of diabetes among human beings. It was found that given proper dose regularly can help reduce the blood glucose levels.

### DETAILED DESCRIPTION OF THE INVENTION

The jamun seeds (dry powder) checks the conversion of starch and carbohydrate into sugar and as such it is best herbal medicine for diabetes. The seeds contain comblian named glucocides, which also checks the conversion of starch into sugar. The glucocides also contains Gerberesin, Gallic acid and other colored components, which are also useful for other treatments.

### EXAMPLE 1

### Treatment of Diabetic Patient with Jamun Stone Powder

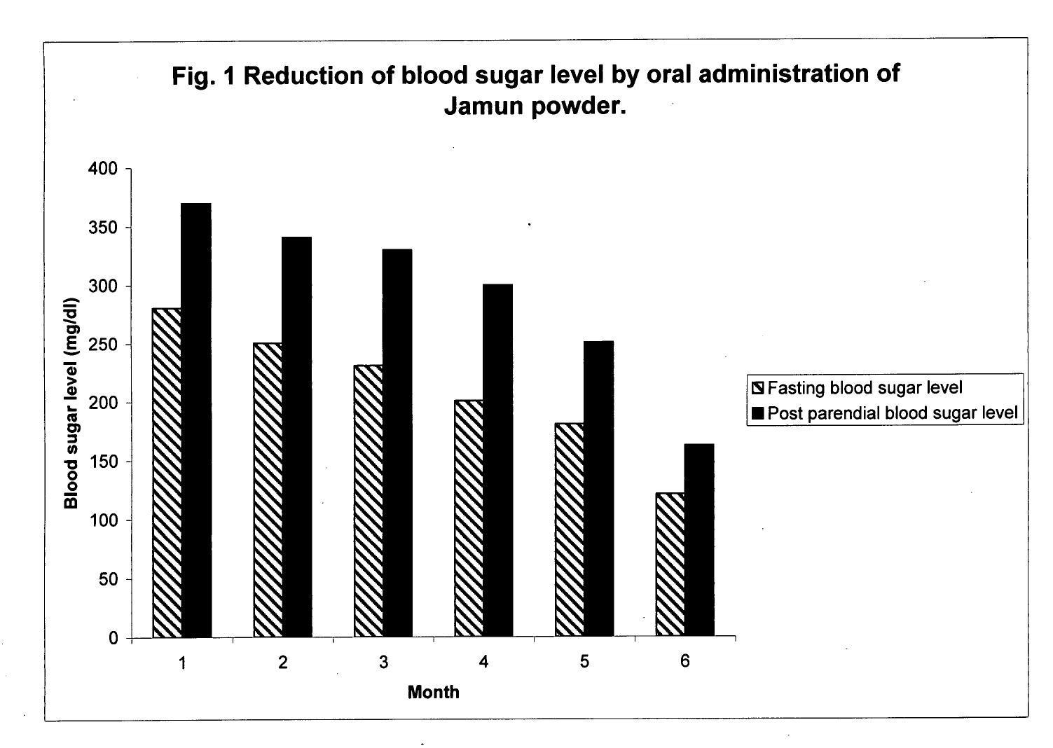

To determine the efficacy of Jamun stone powder in treating diabetic patient, appropriate doses (5-10 grams, two times a day) were given to a 62-year old patient for six months. FIG. 1 and Table 1 show the decrease in blood sugar level over period of time. The data clearly shows that the health of a diabetic patient at initial stage is improved by the decrease in the blood sugar level. The medicine dose of 5-10 gram (atleast) twice a day decreases the fasting blood sugar level [fasting blood sugar level is defined as measuring blood glucose level through standard medical techniques by taking blood samples from the patient who has fasted all night long and such level are usually measured in the morning] by up to 57% percent and decreases the post parendial (PP) blood sugar level [post parendial blood sugar level is defined as measuring blood glucose level through standard medical techniques by taking blood samples from the patient after two hours of him taking food and such level are usually measured in the afternoon] by up to 55%. Please note that fasting and post parendial blood sugar levels were measured the same day as required by standard medical testing protocols. In this study it was proven that the fasting and post parendial (PP) blood sugar levels have been decreased in a six month period at initial stage of diabetes. As per the medical studies, optimum level of fasting blood sugar is 70-110 mg/dl, and optimum level of post parendial blood sugar is 110-160 mg/dl.

TABLE 1
Decrease in blood sugar level of a diabetic patient*.

| | Fasting Blood Sugar | ***Post Parendial Blood |
|---|---|---|
| Month# | Level** (mg/dl) | Sugar (mg/dl) |
| 1^{st} | 280.6 | 369.5 |
| 2^{nd} | 250.5 | 340.6 |
| 3^{rd} | 230.9 | 329.8 |
| 4^{th} | 200.5 | 299.5 |
| 5^{th} | 180.6 | 250.6 |
| 6^{th} | 120.9 | 162.2 |

*Age of Patient: 62 years
**Optimum level of fasting blood sugar level is 70-110 mg/dl
***Optimum level of post parendial blood sugar level is 110-160 mg/dl
#Duration: 6 months
*Dose: 5-10 gm Jamun powder two times a day
**, ***Measurement technique: Standard Medical Testing/Pathological Testing Protocol

As it can be clearly noticed that work related to jamun-extract show anti-diabetic effects, however, such extract had not been carefully studied before and documented for an appropriate dosage to human patients suffering different forms of diabetes. Our work involved efficacy of jamun extraction on human patients and to show the level of effects in reducing diabetes and to prepare a blend that can be medically administered to diabetic patients with no side-effect:

1. Syzigium cumini, (common name, Jamun) is a preventive herbal medicine, which improves the health of a diabetic patient at initial stage by decreasing the blood sugar level.

2. The seeds of Jamun in claim 1 are dried and powdered.

3. The dried powdered form of Jamun in claim 1 is stored at 15 to 40 degree C.

4. The dried powdered form of Jamun in claim 1 checks the conversion of starch and carbohydrate into sugar.

5. The dried powdered form of Jamun in claim 1 contains comblian named glucocides, which checks the conversion of starch into sugar.

6. The dried powdered form of Jamun in claim 1 contains glucocides, comprising of Gerberesin, Gallic acid and other colored components.

7. An oral administration of a dose of dried powdered form of Jamun in claim 1 in the range of 5-10 gm atleast twice a day for a minimum of 6 months is required to prevent diabetes.

8. The dose in claim 7 decreases the blood sugar level by up to 65% (fasting).

9. The dose in claim 7 decreases the blood sugar level by up to 55% (post parendial).
